(19) United States
(12) Patent Application Publication
(10) Pub. No.: US 2008/0206303 A1
(43) Pub. Date: Aug. 28, 2008

(54) Prolonged Release Formulations Comprising Anastrozole

(75) Inventors: Paul Richard Gellert, Cheshire (GB); Balvinder Singh Matharu, Cheshire (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/065,002

(22) PCT Filed: Aug. 29, 2006
(86) PCT No.: PCT/GB2006/003214

§ 371 Date: Feb. 27, 2008

(30) Foreign Application Priority Data
Aug. 31, 2005 (GB) 0517673.0

(51) Int. Cl. A61K 9/00; A61K 31/4196

(52) U.S. Cl. 424/423; 514/383

(57) ABSTRACT

The present invention relates to slow release anastrozole formulations, more particularly to in situ gelling formulations comprising a polylactide polymer or poly(lactide-co-glycolide) co-polymer, in which anastrozole is incorporated. The invention also relates to methods of treatment using said formulations, particularly methods for the treatment of breast cancer, and processes for the preparation of said formulations.

The present invention relates to slow release anastrozole formulations, more particularly to in situ gelling systems, in which anastrozole is incorporated. The invention also relates to methods of treatment using said formulations, particularly methods for the treatment of breast cancer, and processes for the preparation of said formulations.

Anastrozole (Arimidex™) is an aromatase inhibitor, aromatase inhibitors are a class of compounds that act to inhibit oestrogen synthesis in tissues. These compounds prevent oestrogen biosynthesis by inhibiting the enzyme aromatase, which catalyses the conversion of adrenal androgens (androstenedione and testosterone) to oestrogens (oestrogen and oestradiol). Anastrozole is a non-steroidal aromatase inhibitor which is highly selective, well tolerated and is effective in treating advanced breast cancer (Buzdar et al 1995, 5 The Breast 4(3): 256-257 Abs 104; Jonat et al 1995, European Journal of Cancer 32A(3): 404-412; Plourde et al 1995, Journal of Steroid Biochemistry 53:175-179). (Further information on the clinical experience with Arimidex can be found in the prescribing information sheet for Arimidex). Anastrozole is described in U.S. Pat. No. RE 366,717, which is incorporated by reference herein.

The use of injectable implants for the delivery of drugs is well known. Both biodegradeable and non-biodegradeable implant versions have been marketed since the 1980s. Examples of these are Zoladex™, a polylactide-co-glycolide formulation of goserelin for the treatment of breast cancer and Norplant™, a non-biodegradeable silicone device for contraception. Small, injectable microparticle formulations are also well known, an example being Lupron Depot™, a formulation of leuprolide for the treatment of prostate cancer. A drawback of such preformed delivery systems is administration. Cylindrical rods such as Zoladex™ require relatively large bore needles for implantation. Microparticle formulations allow smaller bore needles to be used, however, they require dispersion in an aqueous vehicle prior to injection and their manufacturing processes are typically complex and difficult to control, often involving the use of harsh solvents that require removal. More recently formulations have been developed which are injected as a liquid, but undergo a change to a solid formulation in vivo, so-called ‘in situ gelling systems’. These formulations can be injected subcutaneously through small bore needles and employ only biocompatible solvents. Furthermore, they are relatively simple to manufacture, particularly when compared to microparticle formulations. For a review of such systems, the reader is referred to In Situ Gelling Formulations—Chapter 10, A. J. Tipton and R. L. Dunn. In: Sustained Release Injectable Products, edited by Judy Senior and Michael Radomsky, Interpharm Press, Inc., Buffalo Grove, Ill., 2000. Tipton & Dunn.

Anastrozole is given as a 1 mg tablet daily. It is important that patients take anastrozole daily to receive the optimum therapeutic benefit. Patient compliance with such a daily dosing regimen is however, difficult to ensure, especially where the course of therapy is long or of intermediate or lifetime duration. Thus, there is a need for a prolonged release formulation of anastrozole to improve patient compliance/convenience and give patients optimum therapeutic benefit. Formulating compounds in prolonged release dosage forms, also provides other benefits. For example, less frequent dosing of drugs in the form of prolonged release formulations effectively smoothes out fluctuations in the plasma concentration-time profile. Such smoothing out of plasma profiles has the potential to not only improve the therapeutic effect of the drug, but also to reduce any unwanted side effects. A further advantage of a prolonged release formulation, particularly important for oncology indications, is the improvement in ‘quality of life’ it gives by removing the daily reminder of the disease.

In order to gain patient acceptance of such a formulation over the conventional oral treatment, it is important that treatment with the depot formulation is as comfortable as possible and causes minimal pain on injection. It would be highly advantageous therefore to administer the dose in as small as possible amount of formulation, i.e. low injection volume. In order to achieve this at the oral dose of 1 mg/day, the formulation would need to contain a high weight percentage of anastrozole. However, anastrozole has a molecular weight of 293.4 Daltons and a water solubility of 0.53 mg/ml at 25° C. Low molecular weight compounds with such solubility are not ideally suited to the formation of in situ forming prolonged release formulations. This is because solvent-based depot compositions comprised of a polymer dissolved in a solvent, do not solidify instantaneously after injection. During the initial period following injection where solvent diffuses away from the depot, the rate of diffusion of the active agent is much more rapid than the rate of release that occurs from the subsequently formed solid matrix. Thus, a large percentage of active agent is often released together with the solvent as the system forms. This is particularly evident for low molecular weight compounds that have good aqueous solubility such as anastrozole, which would be expected to rapidly partition out of the depot during the depot formation stage leading to large initial drug bursts and only short periods of drug release. This burst effect is likely to be potentiated still further when formulations contain high drug concentrations and only low levels of rate modifying polymer.

We have worked to develop in situ gelling systems as prolonged release formulations of anastrozole and have surprisingly found that it is possible to produce in-situ gelling formulation for the delivery of anastrozole under certain conditions.

According to a first aspect of the present invention there is provided an in situ gelling formulation comprising:
(i) a polylactide polymer or poly(lactide-co-glycolide) co-polymer having an average molecular weight of from about 10,000 Daltons to about 50,000 Daltons;
(ii) a molar ratio of lactide to glycolide of between about 100:0 to about 50:50;
(iii) from about 5 to about 30% by weight based upon the total weight of the formulation of anastrozole;
(iv) a suitable solvent; and
(v) a polymer to solvent weight ratio of between about 50:50 to about 60:40.
   wherein the in situ gelling formulation continuously releases anastrozole over a period of at least about 7 days when placed in an aqueous physiological-type environment.

According to a further aspect of the present invention there is provided an in situ gelling formulation comprising:
(i) a polylactide polymer or poly(lactide-co-glycolide) co-polymer having an average molecular weight of from about 10,000 Daltons to about 50,000 Daltons;
(ii) a molar ratio of lactide to glycolide of between about 100:0 to about 50:50;
(iii) from about 5 to about 30% by weight based upon the total weight of the implant of anastrozole; and
(iv) a suitable solvent;
(v) a polymer to solvent weight ratio of between about 50:50 to 60:40;
   wherein
(1) For polymer/solvent weight ratios between 50:50 and 55:45
   a) the molar ratio of lactide to glycolide is between 75:25 and 100:0;
   b) when anastrozole loading is above 20% then the molar ratio of lactide to glycolide is between 95:5 and 100:0 and the average molecular weight of the polymer or co-polymer is above 30,000 Daltons;
   c) when the anastrozole loading is 20% or below and the molar ratio of lactide to glycolide is between 75:25 and 85:15 then the average molecular weight of the polymer or co-polymer is above 30,000 Daltons; and
(2) For polymer/solvent weight ratios between 55:45 and 60:40
   a) when anastrozole loading is above 20% then the molar ratio of lactide to glycolide is between 85:15 and 100:0 and the average molecular weight of the polymer or co-polymer is above 20,000 Daltons;
   b) when the anastrozole loading is 20% or below and the molar ratio of lactide to glycolide is between 50:50 and 75:25 then the average molecular weight of the polymer or co-polymer is above 30,000 Daltons.

According to a further aspect of the present invention there is provided an in situ gelling formulation comprising:
(i) a polylactide polymer or poly(lactide-co-glycolide) co-polymer having an average molecular weight of from about 10,000 Daltons to about 50,000 Daltons;
(ii) a molar ratio of lactide to glycolide of between about 100:0 to about 50:50;
(iii) from about 5 to about 30% by weight based upon the total weight of the implant of anastrozole; and
(iv) a suitable solvent;
(v) a polymer to solvent weight ratio of between about 50:50 to 60:40;
   wherein
(1) For polymer/solvent weight ratios between 50:50 and 55:45
   a. the table below gives the appropriate anastrozole loading and average molecular weight of the polymer at the lactide to glycolide ratios given:

| | | Average |
|---|---|---|
| Lactide/Glycolide | Loading | Molecular Weight |
| 75:25-85:15 | >5% | >25,000 |
| 75:25-85:15 | >10% | >30,000 |
| 75:25-85:15 | >15% | >35,000 |
| 86:14-95:5 | >5% | >20,000 |
| 86:14-95:5 | >10% | >25,000 |
| 86:14-95:5 | >15% | >30,000 |
| 95:5 | >20% | >35,000 |
| 96:4-100/0 | >5% | >15,000 |
| 96:4-100/0 | >10% | >20,000 |
| 96:4-100/0 | >15% | >25,000 |
| 96:4-100/0 | >20% | >30,000 |
| 96:4-100/0 | >25% | >40,000 |

and
b. the molar ratio of lactide to glycolide is between 75:25 and 100:0 and when anastrozole loading is above 20% the molar ratio of lactide to glycolide is between 95:5 and 100:0
   and
(2) For polymer/solvent weight ratios between 55:45 and 60:40
   a. the table below gives the appropriate anastrozole loading and average molecular weight at the lactide to glycolide ratios given:

| | | Average |
|---|---|---|
| Lactide/Glycolide | Loading | Molecular Weight |
| 50:50-65:35 | >=5% | >30,000 |
| 50:50-65:35 | >10% | >35,000 |
| 66:34-75:25 | >5% | >20,000 |
| 66:34-75:25 | >10% | >30,000 |
| 66:34-75:25 | >15% | >40,000 |
| 76:24-85:15 | >5% | >15,000 |
| 76:24-85:15 | >10% | >20,000 |
| 76:24-85:15 | >15% | >30,000 |
| 85:15 | >20% | >35,000 |
| 85:15 | >25% | >45,000 |
| 86:14-95:5 | >10% | >15,000 |
| 86:14-95:5 | >15% | >20,000 |
| 86:14-95:5 | >20% | >25,000 |
| 86:14-95:5 | >25% | >35,000 |
| 96:4-100/0 | >15% | >15,000 |
| 96:4-100/0 | >20% | >20,000 |
| 96:4-100/0 | >25% | >30,000 |

and
b. when anastrozole loading is above 20% then the molar ratio of lactide to glycolide is between 85:15 and 100:0

According to a further aspect of the present invention there is provided an in situ gelling formulation comprising:
(i) a polylactide polymer or poly(lactide-co-glycolide) co-polymer having an average molecular weight of from about 10,000 Daltons to about 50,000 Daltons;
(ii) a molar ratio of lactide to glycolide of between about 100:0 to about 50:50;
(iii) from about 5 to about 30% by weight based upon the total weight of the implant of anastrozole; and
(iv) a suitable solvent;
(v) a polymer to solvent weight ratio of between about 50:50 to 60:40;
(vi) wherein
   (1) For polymer/solvent weight ratios between 50:50 and 55:45 the minimum average molecular weight of the polymer can be calculated from the equation below:

MW=83.90−145.3(f_{A}+3.755)+33.96(f_{A}+3.755)²+30.79(f_{G}−0.126)+1822.9(f_{G}−0.126)³

and
(2) For polymer/solvent weight ratios between 55:45 and 60:40 the minimum average molecular weight of the polymer can be calculated from the equation below:

MW=−5.87+43.87(f_{A}+0.078)+182.9(f_{A}+0.078)²+27.99(f_{G}+0.110)+55.0(f_{G}+0.110)²

wherein
f_{A} is the fraction of anastrozole wherein 1.0 is 100%, i.e. 5% would be expressed as 0.05; and
f_{G} is the fraction of glycolide wherein 1.0 is 100%, i.e. 50% would be expressed as 0.50.

The term ‘about’ when relating to the proportion of anastrozole in the formulation refers to ±5% weight percent of the formulation, particularly ±2% weight percent of the formulation.

The term ‘about’ when relating to the duration of release of anastrozole from the formulation refers to +2 days, particularly +1 day, further particularly +12 hours.

The term ‘about’ when relating to the molecular weight of the polymer refers to ±5 kDa, particularly ±2 kDa, further particularly +1 kDa.

The term ‘about’ when relating to ratios of polymer to solvent refers to ±5, particularly ±2, further particularly ±1. It would be clear to the skilled man that ratios are expressed so that the sum of the two figures is always 100. Thus, a ratio of 60/40 could vary between 65/35 and 55/45.

The term “aqueous physiological environment” as used herein refers to the body of a warm blooded animal, particularly man, and especially the subcutaneous environment of such a body. These conditions may be simulated in vitro by placing a formulation in an aqueous dissolution medium, optionally buffered to a physiological pH, at a temperature of from 35 to 40° C. A suitable dissolution medium comprises a saline solution buffered to a pH of approximately 7.4 using a phosphate buffer, for example phosphate buffered saline or McIlvaines citric acid phosphate. Preferably, the aqueous dissolution medium is maintained at a temperature of 37° C.±2° C. The amount of anastrozole released over a given time period may be determined by sampling the dissolution medium and measuring the concentration of anastrozole using a suitable analytical method, for example HPLC.

The term “continuous” as used herein refers to a continual release of anastrozole from the implant for at least 7 days after implantation into an aqueous physiological environment. The rate of release of the anastrozole may vary during the at least 7 day period, for example a short “initial burst” of anastrozole may be observed shortly after implantation followed by a period of lower release. However, there are no periods in the at least 7 days following implantation where the release of anastrozole from the implant is insufficient to maintain in-vivo levels of anastrozole. Preferred levels of release of anastrozole include at least 0.25 mg per day, particularly at least 0.5 mg per day, more particularly about 1 mg of the anastrozole per day when the implant is placed in an aqueous physiological environment. Preferably, the rate of release of the anastrozole is approximately constant, but always continuous, over most of the at least 7 day period.

The term ‘kDa’ refers to kilodaltons.

The term “in situ gelling formulation” as used herein refers to a formulation comprising a drug, a biodegradeable polymer and a biocompatible solvent, which is delivered to a patient as an injectable liquid but solidifies into a solid depot formulation as the liquid solvent diffuses away in vivo.

The term ‘suitable solvent’ refers to any solvent in which the components of the formulation can be dissolved and which after the formulation has been injected in-vivo diffuses from the formulation leading to solidification of the formulation. It is preferred that the solvent for the biodegradable polymer be non-toxic, water miscible, and otherwise biocompatible. Solvents that are toxic should not be used to inject any material into a living body. The solvents must also be biocompatible so that they do not cause severe tissue irritation or necrosis at the site of implantation. Furthermore, the solvent should be water miscible so that it will diffuse quickly into the body fluids and allow water to permeate into the polymer solution and cause it to coagulate or solidify. Examples of such solvents include benzyl alcohol, N-methyl-2-pyrrolidone, 2-pyrrolidone, ethanol, propylene glycol, acetone, methyl acetate, ethyl acetate, methyl ethyl ketone, dimethylformamide, dimethyl sulfoxide, tetrahydrofuran, caprolactam, decylmethylsulfoxide, oleic acid, and 1-dodecylazacycloheptan-2-one. The preferred solvents are N-methyl-2-pyrrolidone and benzyl alcohol.

Formulations of the invention comprise polymers of lactic acid and glycolic acid.

The polylactide polymer is a homopolymer wherein all the repeat units of the polymer are of the Formula (I):

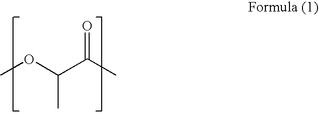

The repeat units are selected from polymers in the D-configuration or a mixture of the L- and D-configurations. Preferably the repeat units of Formula (I) comprise a mixture of L- and D-configurations. When the polymer comprises a mixture of repeat units in the L- and D-configurations the ratio of L- to D-units in the polymer is preferably from 25:75 to 75:25, more preferably from 30:70 to 70:30 and especially approximately 1:1.

Each polymer chain is preferably terminated by one hydroxy group and one —COOH group. However, in embodiments of the present invention other terminal groups may be present, provided that the presence of such terminal groups do not adversely affect the release of anastrozole from the formulation. Suitable terminal groups other than —OH or —COOH which may be present on the polymer include esters formed by reacting an appropriate acid or alcohol with the —OH and/or —COOH end group(s) of the polymer. Suitable esters include alkyl (preferably C₁₋₄-alkyl) or aralkyl (preferably benzyl) esters.

The polylactide polymer may comprise a single polylactide homo polymer or a blend of two or more polylactide homo polymers. A blend of two or more polylactide polymers can be used to provide further control over the rate of release of anastrozole analogue from the formulation, thereby providing a more consistent rate of release over the life-time of the implant in a physiological type environment. Blends of two or more poly(lactide-co-glycolide) polymers or blends of polylactide polymers and poly(lactide-co-glycolide) polymers can also be used. Blends of polymers are particularly useful for minimising “flat spots” in the anastrozole release profile, thereby providing a smooth, steady release of the anastrozole from the formulation.

The polylactide polymer may be prepared using known methods. A preferred method for the preparation of polylactide and poly(lactide-co-glycolide) polymers is ring-opening polymerisation of heterocyclic monomers composed of two lactic or two glycolic acid units, namely, lactide and glycolide, respectively. The ring opening polymerisation is performed under conditions of elevated temperature and in the presence of a suitable catalyst using conditions well known in the polymer art.

Suitable catalysts for the ring-opening polymerisation include but are not limited to zinc, zinc oxide, zinc chloride, p-toluene sulphonic acid, antimony catalysts, for example antinomy trifluoride, or organo-tin catalysts, for example stannous octoate (stannous 2-ethylhexanoate) or tin chloride.

A suitable reaction temperature for the ring-opening polymerisation is from about 120° C. to about 240° C., more preferably from 140° C. to 200° C. The ring opening polymerisation is preferably performed over a period of from 1 to 10 hours, more preferably from 2 to 6 hours.

Preferably the ring opening polymerisation reaction is performed in the presence of a suitable chain termination agent thereby controlling the MW of the resultant polylactide or poly(lactide-co-glycolide) polymer. Suitable chain termination agents include water, a hydroxy-carboxylic acid such as lactic acid or an alcohol, such as a C₁₋₆alkanol. [For further information on suitable methods the reader is referred to standard texts in the art such as: Polylactic and Polyglycolic Acids as Drug Delivery Carriers. L. Brannon-Peppas and M. Vert. In: Handbook of Pharmaceutical Controlled Release Technology, edited by Donald L. Wise, Marcel Dekker, Inc., New York, 2000]

The methods used to prepare polylactide and poly(lactide-co-glycolide) polymers typically results in a mixture of individual polylactide polymer chains, many of which are of differing chain lengths. The polydispersity of a polymer provides an indication of the spread of chain lengths in such a mixture and is defined to be the ratio of the weight average molecular weight (MW) to the number average molecular weight (Mₙ). Suitably, the polydispersity of the polymer is from 1.3 to 4.5.

Formulations of the invention are provided in which a polylactide polymer or poly(lactide/glycolide) copolymer is dissolved in a solvent, which is non-toxic and water miscible, to form a liquid solution. Once the polymer solution is placed into the body where there is sufficient water, the solvent dissipates or diffuses away from the polymer, leaving the polymer to coagulate or solidify into a solid structure. The placement of the solution can be anywhere within the body, including soft tissue such as muscle or fat, hard tissue such as bone, or a cavity such as the periodontal, oral, vaginal, rectal, nasal, or a pocket such as a periodontal pocket or the cul-de-sac of the eye. Anastrozole is added to the polymer solution where it is either dissolved to form a homogeneous solution or dispersed to form a suspension or dispersion of drug within the polymeric solution. When the polymer solution is exposed to body fluids or water, the solvent diffuses away from the polymer-drug mixture and water diffuses into the mixture where it coagulates the polymer thereby trapping or encapsulating the drug within the polymeric matrix as the implant solidifies. The release of the drug then follows the general rules for diffusion or dissolution of a drug from within a polymeric matrix.

In formulations of the invention, the polymer solution is placed in a syringe and injected through a needle into the body. Once in place, the solvent dissipates, the remaining polymer solidifies, and a solid structure is formed. The implant will adhere to its surrounding tissue or bone by mechanical forces and can assume the shape of its surrounding cavity. The degradation time of the implant can be varied depending upon the polymer selected and its molecular weight.

Conveniently the formulation can be stored as two components, consisting of anastrozole and a solution of polylactide polymer or poly(lactide-co-glycolide) co-polymer in solvent. Prior to use the two components are mixed thoroughly. This can be done by storing the two components in syringes. To facilitate mixing the nozzles of the two syringes are connected and the two components thoroughly mixed by pulling the components back and forth between the two syringes. After mixing one of the syringes can be used to dose the formulation, conveniently the dosing syringe can be graduated.

In use, anastrozole is added to the polymer solution prior to injection, and then the polymer/solvent/agent mixture is injected into the body. After injection, the solvent will dissipate and the polymer will solidify and entrap or encase the drug within the solid matrix. Depending on the polymer and solvent used some of the anastrozole may be lost from the formulation as the solvent dissipates, resulting in a burst of anastrozole in-vivo. Such a burst should be kept to a biologically acceptable level, i.e. to minimize any undesired effects of high levels of anastrozole. Burst levels of less than 25-30% burst over first 24 hours are preferred for anastrozole. The release of drug from these solid implants will follow the same general rules for release of a drug from a monolithic polymeric device. The release of drug can be affected by the size and shape of the implant, the loading of drug within the implant, the permeability factors involving the drug and the particular polymer, and the degradation of the polymer. The above parameters can be adjusted by one skilled in the art of drug delivery to give the desired rate and duration of release.

The amount of anastozole incorporated into the injectable, in situ, solid forming formulation depends upon the desired release profile, the concentration of anastrozole required for a biological effect, and the length of time that the drug has to be released for treatment.

The properties of formulations of the invention, in particular the initial release behaviour, can also be varied by changing the weight ratio of polymer to solvent. Preferred polymer solvent ratios depend on the polymers and solvents being used. For example, when using N-methylpyrrolidone as a solvent and lactide/glycolide copolymers between 85:15 and 95:5, ratios of polymer to solvent of between 50:50 and 60:40 are preferred.

Particular novel formulations of the invention include, for example, formulations wherein the characteristics comprise any of the meanings defined hereinafter:—
(a) The solvent is N-methyl-pyrrolidone or benzyl alcohol;
(b) The solvent is N-methyl-pyrrolidone;
(c) The solvent is benzyl alcohol;
(d) The weight average molecular weight of the polylactide or poly(lactide-co-glycolide) polymer is between:
   (i) about 10,000 to about 45,000 Daltons;
   (ii) about 18,000 to about 35,000 Daltons;
   (iii) about 20,000 to about 30,000 Daltons;
   (iv) about 23,000 to about 26,000 Daltons;
      The weight average molecular weight (MW) of the polymer is measured using size exclusion chromatography (SEC) using polymer solutions in Tetrahydrofuran (THF) with 2×30 cm mixed bed ‘D’s PLGel columns (supplier Polymer Laboratories) which have a linear range of MW 200-400,000 Da; wherein the system is first calibrated using PL Easical PS-2 polystyrene calibrants with MW's in the range 580 to 400,000 Da. The PLGel packing material is a highly cross linked spherical polystyrene/divinylbenzene matrix. A Wyatt Optilab DSP refractometer may be used for detection.
(e) The formulation comprises the following proportions of anastrozole:
   (i) about 5 to 25% by weight;
   (ii) about 5 to 20% by weight;
   (iii) about 5 to 15% by weight;
   (iv) about 10 to 25% by weight
   (v) about 10 to 20% by weight
(f) The duration of release is:
   (i) At least about 4 days;
   (ii) At least about 7 days;
   (iii) At least about 10 days;
   (iv) At least about 15 days;
   (v) At least about 21 days;
   (vi) At least about 25 days;
   (vii) At least about 30 days;
   (viii) At least about 40 days;
   (ix) At least about 60 days;
(g) The ratio of polymer to solvent are:
   (i) between about 50/50 and about 60:40;
   (ii) between about 50/50 and about 60:40;
   (iii) about 50/50;
   (iv) about 60/40;
   (v) for N-methyl pyrrolidone—between about 50/50 and about 60:40;
   (vi) for Benzyl alcohol—between about 50/50 and about 60:40;
   (vii) for Benzyl alcohol—about 60:40;
(h) molar ratio of lactide to glycolide is:
   (i) between about 100:0 to about 80:20
   (ii) between about 100:0 to about 90:10
   (iii) about 95:5;
(i) The formulation comprises N-methyl pryrrolidone or benzyl alcohol and has the following characteristics:

| | | | Loading | Duration |
|---|---|---|---|---|
| | | | (about, | of release |
| | | Polymer: Solvent | % by | (at least, |
| L:G ratio | MW | ratio | weight) | about) |
| 75/25 | ≧35 | 50:50 | 5 | 20 |
| 85:15 | ≧30 | 50:50 | 10-20% | 20 |
| 95:5 | ≧26 | 50:50 | 5-10 | 60 |
| D/L 100:0 | ≧30 | 50:50 | 10-15 | 60 |
| 50:50 | ≧35-45 | 60:40 | 10-20 | 30 |
| 65:35 | ≧30-35 | 60:40 | 10-20 | 30 |
| 75:25 | ≧30 | 60:40 | 10-20 | 30 |
| 85:15 | ≧20 | 60:40 | 10-20 | 30 |
| 95:5 | ≧15-20 | 60:40 | 10-20 | 30 |
| D/L 100:0 | ≧10-15 | 60:40 | 10-20 | 30 |

L:G lactide:glycolide
MW molecular weight (about, kDa);

According to a further aspect of the invention there is provided a method for preparing a formulation of the invention comprising
(a) dissolving a polylactide polymer or poly(lactide-co-glycolide) co-polymer in a suitable solvent; and
(b) dissolving an effective amount of anastrozole in the polymer solution.

According to a further aspect of the invention there is provided a method of forming an implant in situ, in a living body, comprising
(a) dissolving a polylactide polymer or poly(lactide-co-glycolide) co-polymer in a suitable solvent;
(b) dissolving an effective amount of anastrozole in the polymer solution;
(c) placing the formulation within the body; and
(d) allowing the solvent to dissipate to produce a solid or gel implant.

### Process a)

Polymer can be dissolved in a suitable solvent by any convenient method such as agitation and mixing. Suitable solvents are as defined above, examples of suitable solvents include N-methyl-2-pyrrolidone and benzyl alcohol.

### Process b)

Anastrozole can be added to the polymer solution in any convenient form such as a powder or dissolved in a suitable solvent. If the anastrozole is dissolved in a suitable solvent this is preferably in the same solvent as used to dissolve the polymer. If anastrozole is added as a powder, it can be dissolved in the polymer solution by any convenient method such as agitation and mixing. To further aid dissolution of anastrozole in the polymer solution, sonication can also be used.

### Process c)

Preferably the formulation is placed in the body by injection at a suitable point in the body. Examples of such sites include the abdomen and the upper buttocks. Preferably the formulation is injected sub-cutaneously.

### Process d)

Upon injection of the flowable formulation, the organic solvent diffuses away from the injection site, causing the polymer to precipitate or gel; thereby entrapping the compound in a sustained-release depot.

According to a further aspect of the invention there is provided a pharmaceutical kit suitable for in situ formation of a biodegradable implant of the invention in the body of a patient, which comprises:
(a) a device containing anastrozole; and
(b) a device containing a solution of a polylactide polymer or a poly(lactide-co-glycolide) co-polymer in a suitable solvent;
   wherein the devices each have an outlet for anastrozole or the polymer solution, an ejector for expelling anastrozole or the polymer solution through the outlet and a hollow tube fitted to the outlet; and wherein the contents of the two devices are mixed together immediately prior to delivering the contents of the device containing the mixture into the body of the patient.

According to a further aspect of the present invention there is provided a medicament comprising a formulation of the present invention.

According to a further aspect of the present invention there is provided a formulation according to the present invention for use as a medicament in the treatment of a condition treatable with anastrozole (preferably breast cancer).

According to a further aspect of the present invention there is provided a method for treating a warm blooded animal (preferably a human) suffering from a condition treatable by anastrozole (preferably breast cancer) comprising administering thereto a formulation according to the present invention.

According to a further aspect of the present invention there is provided a formulation according to the present invention for use as a medicament in the treatment of a condition treatable with anastrozole (preferably breast cancer).

The formulations according to the present invention are useful in the treatment of a wide variety of medical conditions requiring the administration of an aromatase inhibitor, such as anastrozole. Such medical conditions include, but are not limited to, hormone dependent diseases such as breast cancer, ovarian cancer, endometriosis and benign prostatic hypertrophy.

The dose of anastrozole required for the treatment of a particular condition will be dependent upon both the condition being treated and the animal to which it is administered. For example, for the treatment and prevention of breast cancer, the dose of anastrozole is generally 1 mg per day.

According to a further aspect of the present invention, there is provided a method for administering anastrozole to a warm blooded animal, especially a human, comprising injecting (preferably subcutaneously) a formulation according to the present invention in the warm blooded animal.

The invention is further illustrated by the following examples wherein all parts are by weight unless otherwise stated, and the following abbreviations are used:

| | |
|---|---|
| BA | benzyl alcohol |
| AUC | area under the curve |
| NMP | N-methyl-2-pyrrolidone |
| PLGA | poly(lactide-co-glycolide) co-polymer |

### EXAMPLE 1

### PLGA/NMP Liquid Formulation Testing in Rat

Poly(dl-lactide-co-glycolide) was prepared via ring opening condensation of dl-lactide and glycolide dimmers. A quantity of the polymer having a 85/15 ratio of lactide to glycolide, a weight average molecular weight (MW) of 23 kDa and a terminal carboxy group was weighed into a glass sovril bottle and a sufficient amount of pre-sterile filtered NMP was added to give a 60:40 weight ratio of polymer to solvent. The mixture was gently stirred with the aid of a magnetic stirrer bar at room temperature until the polymer completely dissolved. The required amount of anastrozole was then added to the polymer solution and the mixture was sonicated at room temperature to give a clear flowable composition with a 100 mg/ml concentration of drug in solution.

The freshly prepared formulation was filled into 1 ml glass syringes via a 16 gauge blunt needle. The filling needle was then replaced with a one-half inch 21 gauge needle and 100 μl of the polymeric composition was injected subcutaneously into 12 male Wistar rats to give a total dose of 10 mg of anastrozole per rat. The rats were divided into 4 sampling groups to allow blood samples from 3 animals to be collected at each of the following time intervals: baseline, 2, 4, 6, 12, 24 and 36 hours, and days 3, 4, 5, 6, 8, 10, 12, 15, 17, 19, 22, 24, 26, 29, 31, 33, 36, 38, 40, 43, and 46.

Serum samples were assayed for anastrozole using a Liquid Chromatography-tandem Mass Spectrometry method (LC-MS). The serum and percentage cumulative AUC profiles, calculated from the measured anastrozole serum concentrations are shown in FIG. 1. The results show that the formulation released 18% of the drug payload over the first 24 hours. Following this burst, plasma levels remained relatively constant at around 50-150 ng/ml for a duration of 37 days.

### EXAMPLE 2

### PLGA/BA Liquid Formulation in Rat

Poly(dl-lactide-co-glycolide) was prepared via ring opening condensation of dl-lactide and glycolide dimmers. A quantity of the polymer having a 95/5 ratio of lactide to glycolide, a weight average molecular weight (MW) of 26 kDa and a terminal carboxy group was weighed into a glass sovril bottle and a sufficient amount of pre-sterile filtered BA was added to give a 50:50 weight ratio of polymer to solvent. The mixture was gently stirred with the aid of a magnetic stirrer bar at room temperature until the polymer completely dissolved. The required amount of anastrozole was then added to the polymer solution and the mixture was sonicated at room temperature to give a clear flowable composition with a 50 mg/ml concentration of drug in solution.

The freshly prepared formulation was filled into 1 ml glass syringes via a 16 gauge blunt needle. The filling needle was then replaced with a one-half inch 21 gauge needle and 200 μl of the polymeric composition was injected subcutaneously into 12 male Wistar rats to give a total dose of 10 mg of anastrozole per rat. The rats were divided into 4 sampling groups to allow blood samples from 3 animals to be collected at each of the following time intervals: baseline, 2, 4, 6, 12, 24 and 36 hours, and days 3, 4, 5, 6, 8, 10, 12, 15, 17, 19, 22, 24, 26, 29, 31, 33, 36, 38, 40, 43, 46, 49, 52, 55 and 57.

Serum samples were assayed for anastrozole using an LC-MS method. The serum and percentage cumulative AUC profiles, calculated from the measured anastrozole serum concentrations are shown in FIG. 2. The results show that the formulation was capable of achieving continual release of anastrozole for over 56 days.

### EXAMPLE 3

### PLGA/NMP Liquid Formulation Testing in Dog

Poly(dl-lactide-co-glycolide) was prepared via ring opening condensation of dl-lactide and glycolide dimmers. A quantity of the polymer having a 85/15 ratio of lactide to glycolide, a weight average molecular weight (MW) of 23 kDa and a terminal carboxy group was weighed into a glass sovril bottle and a sufficient amount of pre-sterile filtered N-methyl-2-pyrrolidone (NMP) was added to give a 60:40 weight ratio of polymer to solvent. The mixture was gently stirred with the aid of a magnetic stirrer bar at room temperature until the polymer completely dissolved. The required amount of anastrozole was then added to the polymer solution and the mixture was sonicated at room temperature to give a clear flowable composition with a 100 mg/ml concentration of drug in solution.

The freshly prepared formulation was filled into 1 ml glass syringes via a 16 gauge blunt needle. The filling needle was then replaced with a one-half inch 21 gauge needle and 300 μl of the polymeric composition was injected subcutaneously into 4 male Beagle dogs to give a total of 30 mg of anastrozole per dog. Serum samples were collected at baseline, 2, 4, 6, 12, 24 and 36 hours, and days 3, 4, 5, 6, 8, 10, 12, 15, 17, 19, 22, 24, 26, 29, 31, 33, 36, 38, 40, 43, and 46.

Serum samples were assayed for anastrozole using an LC-MS method. The serum and percentage cumulative AUC profiles, calculated from the measured anastrozole serum concentrations are shown in FIG. 3. The results show that the formulation was capable of sustaining the release of anastrozole over a period of 30 days. Following a small drug burst over the first 24 hours (10%), plasma levels remained relatively constant at 20-40 ng/ml throughout the release duration.

### BRIEF DESCRIPTION OF THE DRAWING

FIG. 1 shows the (A) anastrozole in the plasma, and (B) percentage cumulative area under the curve (AUC) profiles following subcutaneous dosing of 100 μl of a 100 mg/ml anastrozole in 60:40 PLGA 85/15 (L/G), 23 kDa:NMP liquid formulation to male Wistar rats wherein the x axis is time in days and in Panel A the y axis is the anastrozole concentration in ng/ml and in Panel B the y axis is percentage cumulative AUC.

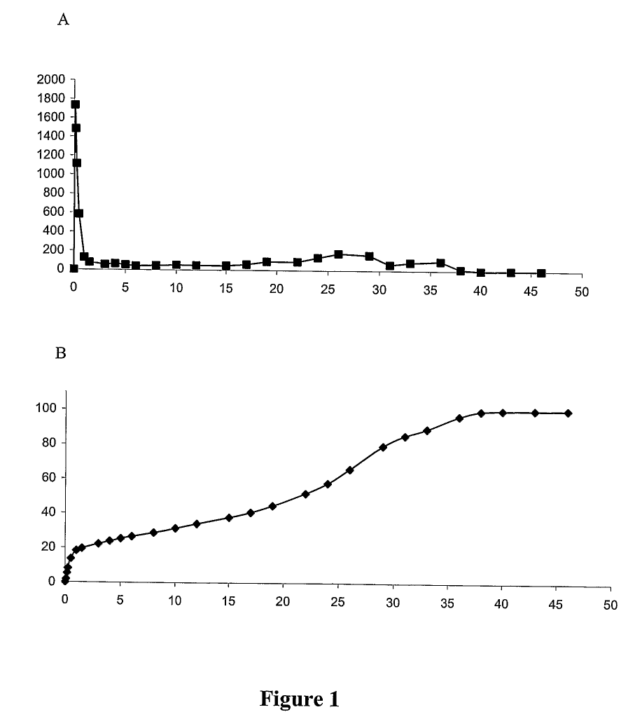

FIG. 2 shows the (A) anastrozole plasma, and (B) percentage cumulative AUC profiles following subcutaneous dosing of 200 μl of a 50 mg/ml anastrozole in 50:50 PLGA 95/5 (L/G), 26 kDa:BA liquid formulation to male Wistar rats, wherein the x axis is time in days and in Panel A the y axis is the anastrozole concentration in ng/ml and in Panel B the y axis is percentage cumulative AUC.

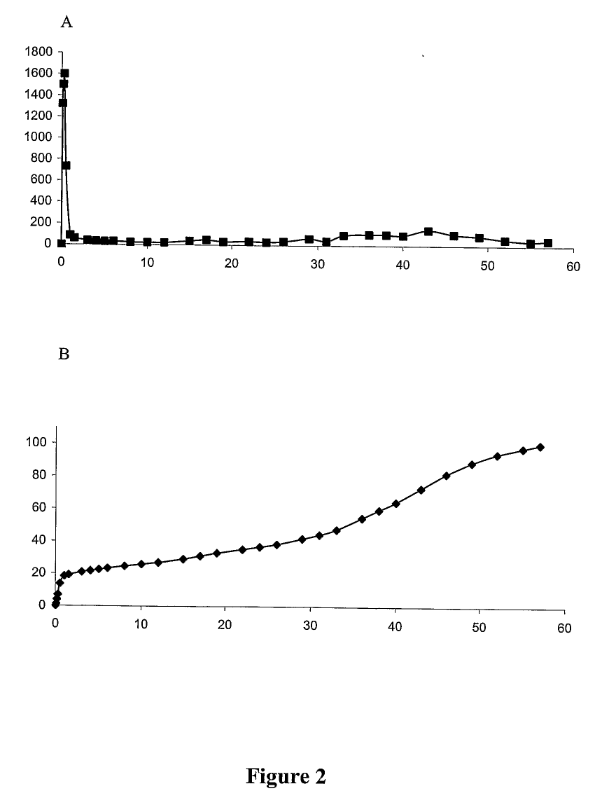

FIG. 3 shows the (A) anastrozole plasma, and (B) percentage cumulative AUC profiles following subcutaneous dosing of 300 μl of a 100 mg/ml anastrozole in 60:40 PLGA 85/15 (L/G), 23 kDa:NMP liquid formulation to male Beagle dogs, wherein the x axis is time in days and in Panel A the y axis is the anastrozole concentration in ng/ml and in Panel B the y axis is percentage cumulative AUC.

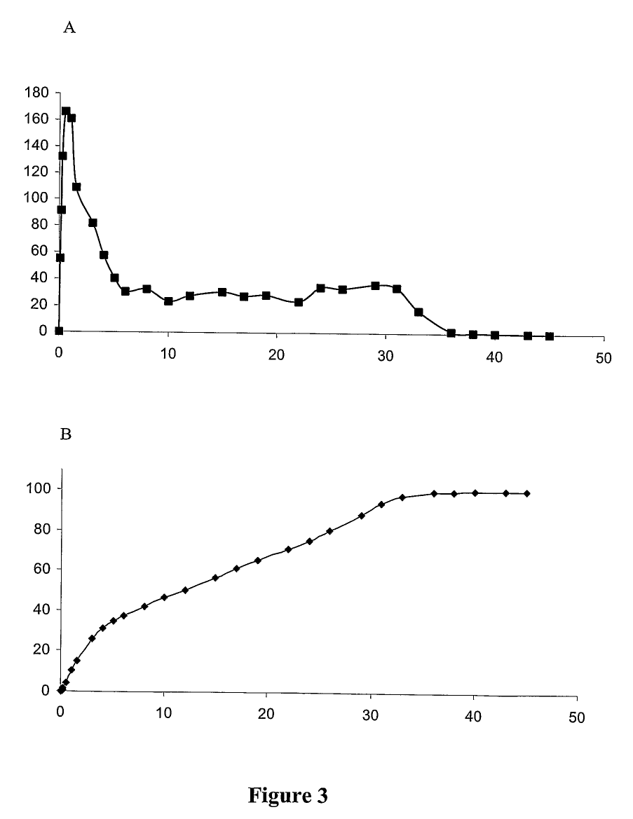

1. An in situ gelling formulation comprising:
(i) a polylactide polymer or poly(lactide-co-glycolide) co-polymer having an average molecular weight of from about 10,000 Daltons to about 50,000 Daltons;
(ii) a molar ratio of lactide to glycolide of between about 100:0 to about 50:50;
(iii) from about 5 to about 30% by weight based upon the total weight of the formulation of anastrozole;
(iv) a suitable solvent; and
(v) a polymer to solvent weight ratio of between about 50:50 to about 60:40;
wherein the in situ gelling formulation continuously releases anastrozole over a period of at least about 7 days when placed in an aqueous physiological-type environment.

2. An in situ gelling formulation comprising:
(i) a polylactide polymer or poly(lactide-co-glycolide) co-polymer having an average molecular weight of from about 10,000 Daltons to about 50,000 Daltons;
(ii) a molar ratio of lactide to glycolide of between about 100:0 to about 50:50;
(iii) from about 5 to about 30% by weight based upon the total weight of the formulation of anastrozole;
(iv) a suitable solvent; and
(v) a polymer to solvent weight ratio of between about 50:50 to about 60:40;
wherein
(1) For polymer/solvent weight ratios between 50:50 and 55:45 the minimum average molecular weight of the polymer can be calculated from the equation below:
MW=83.90−145.3(f_{A}+3.755)+33.96(f_{A}+3.755)²+30.79(f_{G}−0.126)+1822.9(f_{G}−0.126)³
and
(2) For polymer/solvent weight ratios between 55:45 and 60:40 the minimum average molecular weight of the polymer can be calculated from the equation below:
MW=−5.87+43.87(f_{A}+0.078)+182.9(f_{A}+0.078)²+27.99(f_{G}+0.110)+55.0(f_{G}+0.110)²
wherein
f_{A} is the fraction of anastrozole wherein 1.0 is 100%; and
f_{G} is the fraction of glycolide wherein 1.0 is 100%.

3. An in situ gelling formulation according to claim 1 wherein the average molecular weight of the polymer is from about 20,000 Daltons to about 30,000 Daltons.

4. An in situ gelling formulation according to claim 1 wherein the average molecular weight of the polymer is from about 23,000 Daltons to about 26,000 Daltons.

5. An in situ gelling formulation according to claim 1 wherein the molar ratio of lactide to glycolide is about 95:5.

6. An in situ gelling formulation according to claim 1 wherein the anastrozole is between about 5 to about 30% by weight based upon the total weight of the formulation.

7. An in situ gelling formulation according to claim 1 wherein the solvent is selected from N-methyl-pyrrolidone and benzyl alcohol.

8. An in situ gelling formulation according to claim 1 wherein the polymer to solvent ratio is about 50:50

9. An in situ gelling formulation according to claim 1 wherein the polymer to solvent ratio is about 60:40

10. The use of an in situ gelling formulation according to claim 1 as a medicament.

11. The use of an in situ gelling formulation according to claim 1 in the manufacture of a medicament for the treatment of a condition treatable by anastrozole in a warm blooded animal.

12. The use of an in situ gelling formulation according to claim 11 in the manufacture of a medicament for the treatment of breast cancer.

13. A method for preparing an in situ gelling formulation according to claim 1 comprising
(a) dissolving a polylactide polymer or poly(lactide-co-glycolide) co-polymer in a suitable solvent; and
(b) dissolving an effective amount of anastrozole in the polymer solution.

14. A method of forming an implant in situ, in a living body, comprising
(a) preparing an in situ gelling formulation according to the method of claim 13;
(b) placing the formulation within the body; and
(c) allowing the solvent to dissipate to produce a solid or gel implant.

15. A pharmaceutical kit suitable for in situ formation of a biodegradable implant, from a in situ gelling formulation according to claim 1, in the body of a patient, which comprises:
(a) a device containing anastrozole; and
(b) a device containing a solution of a polylactide polymer or a poly(lactide-co-glycolide) co-polymer in a suitable solvent;
wherein the devices each have an outlet for anastrozole or the polymer solution, an ejector for expelling anastrozole or the polymer solution through the outlet and a hollow tube fitted to the outlet; and wherein the contents of the two devices are mixed together immediately prior to delivering the contents of the device containing the mixture into the body of the patient.

16. An in situ gelling formulation according to claim 2 wherein the average molecular weight of the polymer is from about 20,000 Daltons to about 30,000 Daltons.

17. An in situ gelling formulation according to claim 2 wherein the average molecular weight of the polymer is from about 23,000 Daltons to about 26,000 Daltons.

18. An in situ gelling formulation according to claim 2 wherein the molar ratio of lactide to glycolide is about 95:5.

19. An in situ gelling formulation according to claim 2 wherein the anastrozole is between about 5 to about 30% by weight based upon the total weight of the formulation.

20. An in situ gelling formulation according to claim 2 wherein the solvent is selected from N-methyl-pyrrolidone and benzyl alcohol.

21. An in situ gelling formulation according to claim 2 wherein the polymer to solvent ratio is about 50:50.

22. An in situ gelling formulation according to claim 2 wherein the polymer to solvent ratio is about 60:40.

23. The use of an in situ gelling formulation according to claim 2 as a medicament.

24. The use of an in situ gelling formulation according to claim 2 in the manufacture of a medicament for the treatment of a condition treatable by anastrozole in a warm blooded animal.

25. The use of an in situ gelling formulation according to claim 24 in the manufacture of a medicament for the treatment of breast cancer.

26. A pharmaceutical kit suitable for in situ formation of a biodegradable implant, from a in situ gelling formulation according to claim 2, in the body of a patient, which comprises:
(a) a device containing anastrozole; and
(b) a device containing a solution of a polylactide polymer or a poly(lactide-co-glycolide) co-polymer in a suitable solvent;
wherein the devices each have an outlet for anastrozole or the polymer solution, an ejector for expelling anastrozole or the polymer solution through the outlet and a hollow tube fitted to the outlet; and wherein the contents of the two devices are mixed together immediately prior to delivering the contents of the device containing the mixture into the body of the patient.
